(19) United States
(12) Patent Application Publication
(10) Pub. No.: US 2005/0077459 A1
(43) Pub. Date: Apr. 14, 2005

(54) Radiation phantom

(76) Inventors: Mark J. Engler, Lexington, MA (US); Mark J. Rivard, Hopkinton, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/783,313

(22) Filed: Feb. 19, 2004

Related U.S. Application Data
(60) Provisional application No. 60/448,255, filed on Feb. 19, 2003.

(51) Int. Cl. G21K 1/12; H05G 1/60; A61B 6/00; G01N 23/00; G12B 13/00; G01D 18/00

(52) U.S. Cl. 250/252.1

(57) ABSTRACT

A real, physical radiation phantom for simulating a portion of a human being includes a body portion providing an analytic outer shape of the phantom, the outer shape being similar to a shape of at least a portion of the human being, the body portion having a first physical characteristic of a first value similar to a second value of the first physical characteristic corresponding to human soft tissue, and at least one internal component disposed in the body, the internal component having an analytic shape approximating an internal portion of human anatomy and having a third value of the first physical characteristic different from the first value.

### CROSS-REFERENCE TO RELATED ACTIONS

This application claims the benefit of U.S. Provisional Application No. 60/448,255 filed Feb. 19, 2003 that is incorporated here by reference.

### FIELD OF THE INVENTION

The invention relates to radiation phantoms including systems of real and virtual radiation phantoms.

### BACKGROUND OF THE INVENTION

Radiotherapy allows radiation oncologists to treat medical conditions including cancerous and other tumors and neoplastic tissues within a patient's body. Radiotherapy often serves as an alternative to more invasive surgical procedures and other therapies such as chemotherapy, that have increased risk of adverse side effects including death. Radiotherapy is delivered with external beams of ionizing photons, electrons, and other particulate radiation, and with radioactive sources placed in body cavities or with thin needles interstitially. Ionization of the radiotherapy target inhibits cell growth by sterilizing cells and their progeny.

Modern radiotherapy is planned with computers that simulate three-dimensional dose distributions arising from diverse arrangements of external beams or internal sources. Modern external beam radiotherapy (EBRT) is delivered with diverse collimators, such as multi-leaf collimators, and other devices that form small beams (beamlets) with precisely known geometric and physical characteristics describing their deposition of dose in tissues. The beamlet cross section perpendicular to the central ray may have dimensions on the order of 1 mm. This corresponds to the spatial resolution of computerized tomographic and magnetic resonance imaging applied to identify in the radiation treatment planning system (RTPS) the treatment planning target volume (PTV) and surrounding healthy tissues, or organs at risk (OAR). Radiotherapy aims to deliver a maximum radiation dose to the PTV that will control or cure the target, while minimizing dose to OAR to minimize adverse side effects. Radiotherapy tries to maximize the ratio of biological dose to the PTV divided by the biological dose to the OAR, referred to as the therapeutic ratio. Intensity modulated radiation therapy (IMRT) and radiosurgery (SRS) are two examples of external beam radiation therapy that utilize many beamlets to create physical dose distributions precisely conforming to the target so as to maximize the therapeutic ratio. Currently in the field of IMRT, maximizing the therapeutic ratio is achieved by searching the phase space of all possible beamlets to find minimum values of objective functions that contain terms for maximizing PTV dose while minimizing OAR dose.

A major challenge in the field of IMRT is to design a clinical phase III trial proving that the enhanced precision of IMRT and the intelligence of its computer treatment planning will translate into clinical results superior to those of other attempts. IMRT delivery and planning devices have proliferated in a rapid and highly creative fashion. Consequently the practice of IMRT, even when limited to only one of many commercial systems, involves dose distributions with wide ranging characteristics that are unable, within an affordable amount of time, to develop the statistical power needed to prove clinical superiority. Because of this lack of proof of clinical advantage, IMRT reimbursement is being challenged to an extent where IMRT research and development may be severely impaired.

In addition to the challenges of disparate practice of IMRT, modern radiotherapy systems are challenged by innate uncertainties in value and location of dose, i.e., in the dose distribution. Geometric uncertainties include those of beam or source geometry and those of dose calculation. Beam geometry uncertainties stem from imperfections in collimating and other systems of the external beam machine, e.g. a medical linear accelerator (linac), and from diverse patient motions and deformations during a treatment. Source geometry uncertainties stem from errors in imaging the sources and patient motion. Dose calculation uncertainties stem from errors in modeling the many absorption processes involved in diverse tissue compositions and geometries, and from errors in propagating uncertainties of beam and source geometry.

Currently the quality assurance of modern radiotherapy technology and practice is as disparate and complicated as the technology and practice themselves. In typical quality assurance of the integrity of radiation treatment planning systems, a regular shaped phantom, simulating certain properties of human tissues, is imaged and entered into planning software. Beam or source intensity distributions are then applied to calculate dose distributions in the phantom. The phantom is then embedded with a detector so that a measurement can be made to verify the calculated dose. Most often only a dose point in a region of uniform dose is measured to spot check an entire dose distribution. However, the most clinically critical component of the dose distribution consists of regions of high dose gradient between the target and adjacent normal tissues, where discrepancies between modeled and measured doses are most likely to be found. These discrepancies are likely to be greatest when the high gradient region exists in heterogeneous tissues, e.g., including bone, soft tissue, and air, as in the sinuses and other body passages and cavities associated with breathing.

Real and virtual phantoms have been used previously for quality assurance and/or testing for radiation therapy and nuclear medicine. The primary anthropomorphic phantom used in radiation therapy for approximately the last 40 years has been the Alderson-Rando phantom. Alderson, Lanl, Rollins, Spira, Am. J. Roentgenol. 87, pp. 185-195 (1962). This phantom uses an embedded human skeleton such that skeletal geometry varies from phantom to phantom and the phantom is not characterized by analytic equations. Virtual phantoms using analytically-defined components are exemplified by the MIRD phantom that has been applied in the field of nuclear medicine. Snyder, Ford, Warner, “Estimates of Specific Absorbed Fractions for Photon Sources Uniformly Distributed in Various Organs of a Heterogeneous Phantom,” MIRD Pamphlet No. 5, Revised, Society of Nuclear Medicine (New York, N.Y.) January 1978, pp. 5-67. Recently, a modified MIRD head phantom has been used in neutron capture therapy. Goorley, Kiger, Zamenhof, “Reference Dosimetry Calculations for Neutron Capture Therapy with Comparison of Analytical and Voxel Models,” Med. Phys. 29 (2), February 2002, pp. 145-156.

### SUMMARY OF THE INVENTION

Embodiments of the invention provide improved phantoms and techniques for phantom use. Embodiments of the invention integrate real and virtual phantom features into a system of physical, real phantoms (RPs) and virtual phantoms (VPs), e.g., to facilitate implementation of highly-sophisticated radiation technologies such as IMRT. Such systems are referred to as real-virtual phantom (RVP) systems, and these systems can be applied to a wide array of applications including dosimetric modeling and verification, external radiation applications (e.g., EBRT), and internal radiation applications (e.g., brachytherapy).

In general, in an aspect, the invention provides a real, physical radiation phantom for simulating a portion of a human being, the phantom including a body portion providing an analytic outer shape of the phantom, the outer shape being similar to a shape of at least a portion of the human being, the body portion having a first physical characteristic of a first value similar to a second value of the first physical characteristic corresponding to human soft tissue, and at least one internal component disposed in the body, the internal component having an analytic shape approximating an internal portion of human anatomy and having a third value of the first physical characteristic different from the first value.

Implementations of the invention may include one or more of the following features. The first physical characteristic is one of density and effective atomic number. The at least one internal component is configured to approximate a shape of a human bone and the third value is one of an average density of the human bone and an effective atomic number of the human bone. The at least one internal component is configured to approximate a shape of a human bone, at least a part of the at least one internal component including multiple portions configured to simulate different layers of bone, the multiple layers including a first portion having a first density and a first atomic number similar to a density and atomic number of an outer, relatively harder layer of human bone and a second portion inside the first portion and having a second density and a second atomic number similar to a density and atomic number of an inner, relatively softer layer of human bone. The at least one internal component comprises multiple internal components of shapes approximating internal components of the human being and having corresponding densities and atomic numbers similar to the corresponding internal components of the human being. The multiple internal components have densities and atomic numbers similar to at least one of bone, soft tissue, lung, and fat. The phantom provides at least one hole configured to receive a radiation detector and sized to permit rotation of the radiation detector inside the phantom. The phantom provides at least one passage extending from an outer surface of the body to a cavity defined inside the phantom, the passage being configured to convey at least one of gas and liquid to the cavity.

In general, in another aspect, the invention provides a real-virtual phantom system including an anthropomorphic virtual phantom that includes analytic shapes representing human anatomical parts, and an anthropomorphic real, physical phantom that approximates the virtual phantom in a radiation-relevant manner with a first material that simulates human soft tissue and at least one second material that simulates other tissue that affects radiation differently than soft tissue, the at least one second material having an analytic shape that approximates a corresponding portion of human anatomy.

Implementations of the invention may include one or more of the following features. Corresponding portions of the virtual and real phantoms have similar densities and atomic numbers. The densities and atomic numbers correspond to at least one of bone, soft tissue, lung, and fat. The real phantom provides at least one hole configured to receive a radiation detector and sized to permit rotation of the radiation detector inside the phantom. The real phantom provides at least one passage extending from an outer surface of the real phantom to a cavity defined inside the real phantom, the passage being configured to convey at least one of gas and liquid to the cavity. The anthropomorphic virtual phantom comprises numerical expressions disposed on a computer-readable medium. The analytic shapes of human anatomical parts of the anthropomorphic virtual phantom represent human anatomical parts that are high-probability targets for radiation therapy.

In general, in another aspect, the invention provides a method of using a first virtual radiation phantom, the method including calculating a first radiation distribution from a first radiating device in the first virtual radiation phantom, the first virtual radiation phantom modeling human anatomical components as analytic shapes, and comparing indicia of the first radiation distribution with information from a second radiation distribution.

Implementations of the invention may include one or more of the following features. The information from the second radiation distribution is information of radiation detected in a first physical phantom configured to approximate physical characteristics modeled by the first virtual radiation phantom in a radiation-relevant way. The first virtual radiation phantom and the physical phantom are substantially similar to a second virtual radiation phantom and a second physical phantom used with a second radiating device as part of a clinical test. The method further includes troubleshooting the first radiating device if appropriate as determined from the comparing. The method further includes radiating a human patient and providing information associated with radiating the human patient to a repository of information for a clinical test. The radiating comprises radiating the patient with an IMRT device. The method further includes performing an analysis on at least one of the indicia of the first radiation distribution and the information from the second radiation distribution and adjusting radiation parameters of the first radiating device, if appropriate, based upon the analysis. The information from the second radiation distribution is information calculated using a Monte Carlo radiation transport analysis.

Various aspects of the invention may provide one or more of the following capabilities. A universally-accepted quality management system (QMS) can be provided to pave the way towards clinical trials with standardized and verifiable dosimetric criteria that would encourage accelerated patient accrual, proof of clinical value, and standards of practice for facilities not involved in clinical trials. Evolving standards may include per-patient dosimetric verification in the RVP system. Verification may extend from point dose spot checks to comparisons between modeled and actual radiation dose distributions determined from a multiplicity of measurements. Modifications can be made in radiation delivery parameters to compensate for discrepancies between modeled and measured dose distributions. Effects of mechanical positioning errors, e.g., due to gantry sag or table wobble, may be detected and the errors corrected. RPs can be produced less expensively than previous phantoms and can provide more consistent tissue density distributions than previous phantoms.

Variations in normal human anatomy and complex target shapes can be easily simulated in VPs. VPs can be transported electronically without compatible CT (computer tomography) image data sets that may require study-subject de-identification and institutional board study review. VPs can be stored and manipulated with less computer memory than currently occupied by CT image data sets. Benchmark treatment plans can be generated and RTPSs evaluated. Dosimetry of IMRT and other radiotherapeutic modalities and systems can be verified. The technical expertise needed for a clinical facility to be credentialed for nationally-coordinated clinical trials, or accredited for standards of care required by regulatory bodies such as state governments, may be demonstrated. Direct comparisons of the RTPS calculation can be made with more accurate (than typical RTPS calculations), “gold standard” Monte Carlo radiation transport analysis if appropriate Monte Carlo hardware and software are available. Multiple facilities can compare radiation distributions against common RVP embodiments that have similar characteristics (e.g., identical VPs and separate RPs made to the same VP specifications). RVP systems can better simulate human anatomy than previous RPs and can be produced repeatably so that different facilities can use separate phantoms with similar characteristics. Dosimetric criteria and protocols are more likely to be standardized across different facilities and different radiation systems with the advent of RVP systems.

These and other capabilities of the invention, along with the invention itself, will be more fully understood after a review of the following figures, detailed description, and claims.

### BRIEF DESCRIPTION OF THE FIGURES

FIG. 1 is a simplified block diagram of a radiation therapy system.

FIGS. 2-4 are perspective views of exemplary VPs.

FIG. 5 is a two-dimensional cross-sectional view of an exemplary VP of male pelvic anatomy.

FIGS. 6-12 are graphs and equations of location and size variations of components of the VP anatomy shown in FIG. 5 as a function of cranial-caudal position.

FIG. 13 is a simplified perspective view of an RP made in accordance with the specifications of the VP shown in FIG. 5.

FIG. 14 is a block flow diagram of a process of producing virtual and real radiation phantoms.

FIG. 15 is a block flow diagram of a process of accepting/commissioning a radiating device of the radiation therapy system shown in FIG. 1.

### DETAILED DESCRIPTION OF PREFERRED EMBODIMENTS

Embodiments of the invention provide techniques for testing and analyzing performance of radiating devices and for benchmarking and/or standardizing the provision of radiation. Systems, called Real-Virtual Phantoms or RVPs, of VPs and corresponding RPs are used in the testing and/or analyzing of radiating device performance, benchmarking, and/or standardizing. An anthropomorphic VP (e.g., a mathematical/numerical description of a phantom) composed of analytic geometric shapes simulates, in a simplified manner, the anatomy of a human at least in a region that is to receive radiation. A radiation distribution in the VP can be calculated based upon the shapes of components of the VP, physical characteristics (e.g., density distribution) of the components, and radiation parameters of a radiating device to apply the radiation. A real, physical anthropomorphic phantom is also produced and/or supplied that closely approximates the VP's component shapes and characteristics. Radiation is applied to the RP and the induced radiation distribution is detected. The calculated and measured distributions of the virtual and real phantoms can be compared to determine deviations between the two. Troubleshooting can be undertaken to attempt to rectify causes of undesirably high disparities between the two distributions. Analysis of either of the distributions may be undertaken to determine adjustments to be made to the radiating device and/or radiation plan implemented by the radiating device. The VP can be provided, e.g., electronically, for use with different radiating devices. Also, multiple RPs with similar characteristics and shapes of components can be provided to and used with the different radiating devices. This can be done to provide a standard such that different devices/facilities can meaningfully compare their results with each other and can provide a reference point for the devices. Thus, embodiments of the invention can help ensure commonality/standardization of radiating devices such that equal indications of applied radiation from different devices in fact indicate equal absolute amounts of applied radiation. Thus, for example, embodiments of the invention may help IMRT be used in clinical trials, e.g., to verify whether IMRT provides a superior technique for radiation therapy. Other embodiments are within the scope of the invention.

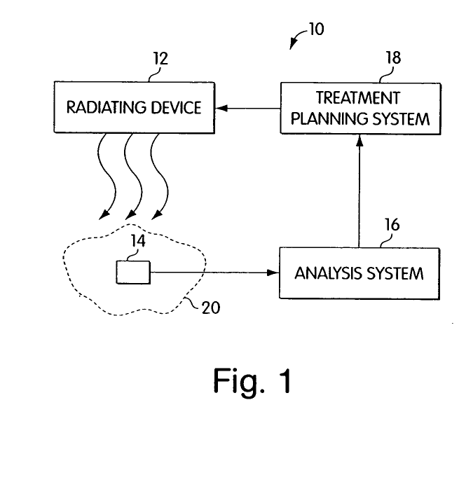

Referring to FIG. 1, a radiation system 10 includes a radiating device 12, a radiation detector 14, a profile or dose distribution analysis system 16, and an RTPS 18. The radiating device may be any of a variety of devices such as a Gamma Knife, a linear accelerator (linac), or radioactive sources. The radiating device 12 is configured to radiate portions of a volume 20 (that may be a phantom or a patient, or portions thereof) and the analysis system 16 (e.g., including a controller and a positioning device similar to those described above) is configured to detect and analyze the radiation provided by the device 12.

The treatment planning system 18 can be used by a radiation oncologist to determine/develop a treatment plan and/or used by a qualified radiation physicist to develop a test plan. Based on desired characteristics of the radiation, the treatment planning system 18 determines how to configure the radiating device 12 and determines expected radiation distribution values. The distribution preferably maximizes radiation to a target region (e.g., a tumor) and minimizes radiation to regions outside the target region, e.g., healthy surrounding tissue for treatment plans. Determined treatment plans can be applied to patients or phantoms, e.g., a VP or an RP. Determined test plans are applied to VPs and verified in RPs.

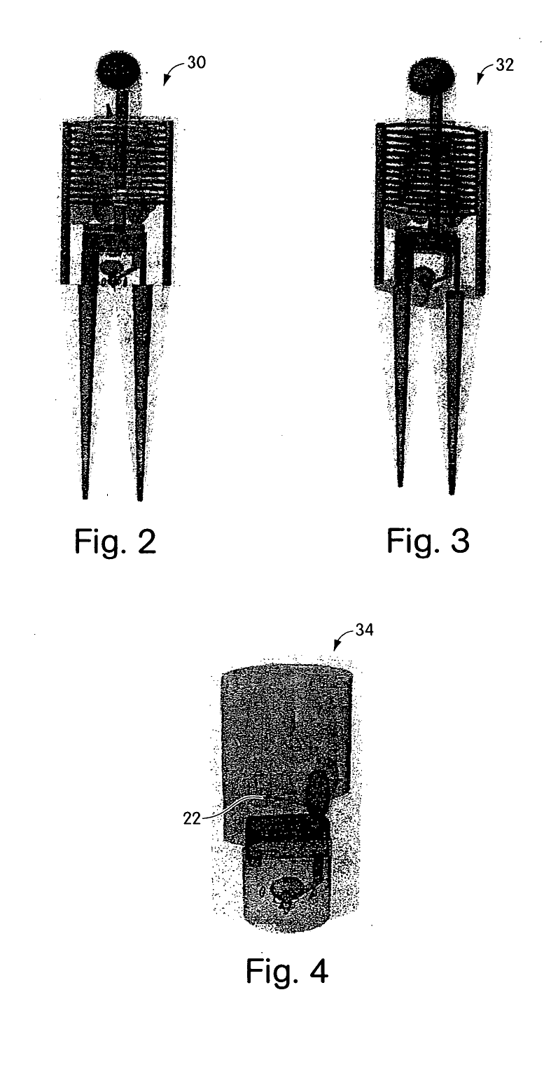

Referring to FIGS. 2-4, various anthropomorphic VPs can be produced, e.g., by a radiation oncologist and/or a qualified medical physicist, etc. for use in testing the system 10. FIG. 2 shows a full-body VP 30 of a human female. FIG. 3 shows a full-body VP 32 of a human male. FIG. 4 shows a pelvis VP 34 of a human female. Different portions of humans or other animals (e.g., models of a head, neck, or prostate anatomy), or other objects (living or not) can be approximated in phantoms as desired. Diverse, complex, and realistic target shapes, and a range of sizes may be approximated in phantoms as desired. The VPs 30, 32, 34 preferably model at least bone, soft tissue, and lungs with different properties (e.g., density and/or effective atomic number). The phantoms 30, 32, 34 may model other components differently, e.g., fat. Further, the VPs 30, 32, 34 model anatomical components that are commonly targeted (i.e., high-probability targets) for radiation therapy, e.g., parts of the anatomy that frequently develop cancer such as the prostate.

As shown, the VP is a collection of analytic geometric shapes selected, sized, and arranged to approximate an anatomy of interest. Virtual radiation phantoms preferably use three-dimensional solids, such as spheres, cylinders, cones, ellipsoids, parallelepipeds, etc. described by geometric equations. For example, if it is desired to verify radiation dose distributions provided by the radiating device 12 to a patient's pelvis, the oncologist and/or other appropriate person(s) (assumed below to be just the oncologist) geometrically models a human pelvis. The oncologist uses/derives mathematical equations relating to pelvic geometry and relating to the geometry of the anatomic components within the male pelvic anatomy, such as the bladder, prostate, and rectum. The oncologist further develops one or more equations to represent/model a radiation target 22, such as a lesion or tumor. The VPs 30, 32, 34 shown are exemplary, and variations of these phantoms may be used. For example, some of the components shown may not be modeled, and other components may be added to the VPs shown. More complex modeling may also be used. For example, while bones are shown as being modeled as cylinders or cones alone, they may be modeled with multiple geometries and may include joints, such as knees. Further, these or other components may be described by more than one equation or numerical model, including being split into multiple pieces with different geometric equations and/or other characteristics (e.g., density and/or atomic number). Based upon the mathematical equations and other appropriate information, the oncologist generates a VP of the male pelvic anatomy, e.g., the phantom 34 shown in FIG. 4.

The different portions of the phantoms have properties corresponding to the portion of the anatomy/object that they represent. For example, the densities of the various components in the phantoms 30, 32, 34 are preferably chosen to correspond with the densities of organs in a human body, and torso, respectively. The physical properties of the VP components are thus similar to those of the object (e.g., human) that the VPs represent. In particular, bone is preferably modeled in the VPs 30, 32, 34 to have a density equal to the average density of human bone and an atomic number equal to the effective atomic number (that affects radiation absorption) of human bone. The density and atomic number of the portions of the VPs are known. The density and/or atomic number may be uniform throughout a VP component or may be non-uniform within a VP component.

Parts of the VP can be modified to reflect conditions that may exist in a patient, including normal, healthy conditions and/or abnormal and/or unhealthy conditions. For example, virtual lungs or other cavities can be filled with substances such as gases to various pressures. Cavities may also be filled with other substances such as fluid at least partially filling a lung to reflect an unhealthy lung, possibly indicating disease in the lung. One or more gases and one or more fluids could be put into a common cavity in the phantom. Different compositions may be modeled to provide a desired density and/or atomic number of material in a desired location in the VP.

One or more portions of the VP may be modeled in greater detail if desired. For example, for tumors in or around the spinal column, the bone of the spine in the region of the tumor may be modeled in more detail. The anatomy may be modeled down to the millimeter level. The bone is preferably modeled in its respective layers, e.g., the dura (hard outer layer), the inner (soft) layer, and the marrow if the bone in consideration has marrow. The bone layers may be modeled, e.g., the hard bone, the soft bone, and the marrow with specific gravities of, e.g., about 1.8, 1.3, and 1.0, respectively.

The equations relating to the geometric VP models are stored in computer storage, such as a database, of the treatment planning system 18. The stored values provide a library of geometric modeling equations. The database allows the oncologist to retrieve earlier modeled geometries in the library and alter the parameters or equations in order to simulate variations in human anatomy or to simulate variations in target (e.g., lesion or tumor) shapes and sizes. Furthermore, by storing the equations in a database, geometric models for particular anatomical portions (e.g., male pelvic anatomy) can be electronically transported to other facilities such that multiple facilities can use identical VPs. The VPs can be electronically transported without transporting a computer tomography (CT) image dataset of a phantom. Transmission of such CT datasets can require patient de-identification with respect to the dataset or board review of the data set, both processes being relatively time intensive. Storage and manipulation of the geometric model equations by a computer system can use relatively less computer resources and memory, as compared to computer system storage and manipulation of the CT datasets.

The treatment planning system 18 can use the VP models to computer radiation distributions. The system 18 uses the equations of the various components of the models, and their corresponding densities to compute radiation distributions based on radiation parameters and the physical effect of the various components on the incident radiation. Thus, the system 18 can account for scattering effects, attenuation effects, etc. of the VP that are similar to the effects of the object (e.g., portion of a human) that the VP simulates.

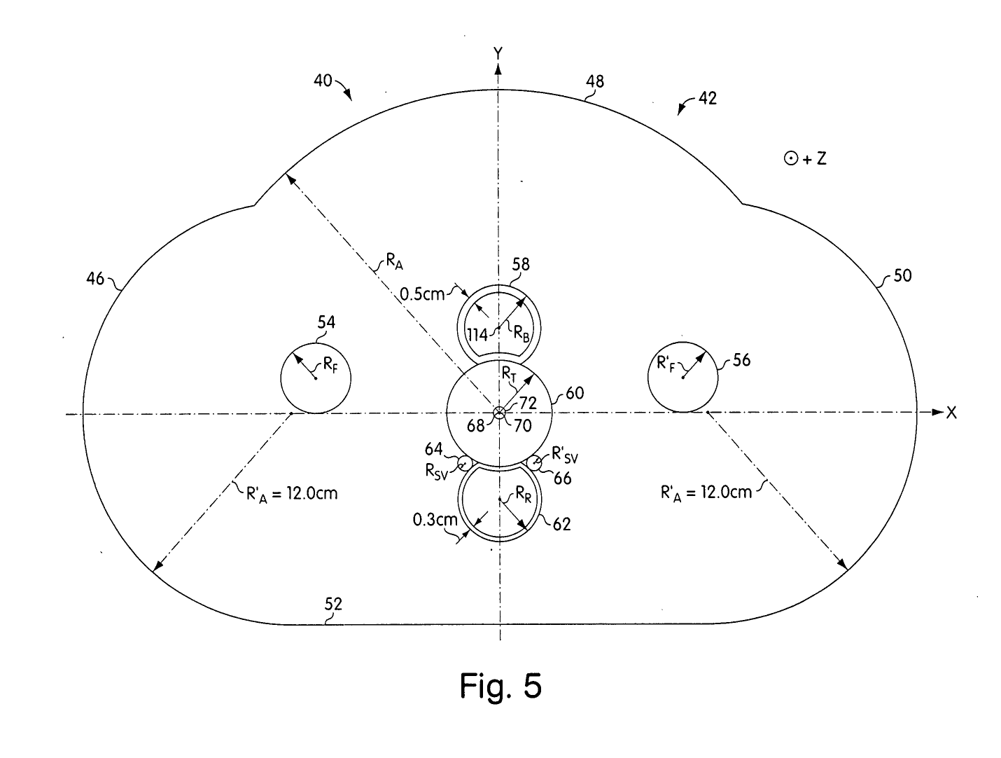

Referring to FIG. 5, a virtual phantom geometric model 40 of a male pelvic anatomy 42 contains various simulated anatomical components 44. The model 40 shown is exemplary only and not limiting. The illustrated model 40 is a two-dimensional cross-section in a plane at +130.3 mm in z that is parallel to but displaced from the x-y plane, and is thus transverse to the cranial-caudal axis, here the z-axis (going into and coming out of the page). Femoral heads and a bladder are projected to this plane because in this exemplary model they do not exist in the plane at the +130.3 mm along the z-axis. The model 40 of the torso 42 in the two-dimensional cross-section shown is a union of three circles 46, 48, and 50 and the edge of a rectangle 52 to approximate the contour of a human pelvis.

The geometric components 44 are located within the male pelvic anatomy 42 to simulate male pelvic anatomy. The components 44 are designed to simulate the effects that corresponding pieces of anatomy will have on the radiation dose distribution within the torso 20 when exposed to radiation. The components 44 are represented by analytic functions/equations. As shown, the model 40 includes two femoral heads 54, 56, a bladder 58, a prostate 60, a rectum 62, two seminal vesicles 64, 66, and a urethra 72. The anatomical components 44 here are modeled as circles in cross-section, each circle having an associated radius. The bladder 58 and the rectum 62 are discs formed of an inner and an outer circle. Thus, the bladder 58 and the rectum 62 in three dimensions are cavities with the bladder 58 having an exemplary wall thickness of 0.5 cm and the rectum 62 having an exemplary wall thickness of 0.3 cm. Where the bladder 58 or rectum 62 interfere with other components (here, the prostate 60), they deform as shown, being pushed inwardly, taking the shape of the prostate 60. The location of the centers of the circles and/or the sizes of their radii may vary in three dimensions such that in different cross-sections, the sizes and/or locations of the components 44 may be different than as shown in FIG. 5.

As shown, a center point 68 of the torso 42 is disposed at the origin of the coordinate system shown, i.e., at the intersection of the x-axis, y-axis, and z-axis. The center point 68 is taken as the center of the circle 48. The origin is also coincident with a center point 70 of the prostate 60.

The geometric model 40 shown in FIG. 5 illustrates two-dimensional relationships (e.g., along the x-axis and y-axis) among the anatomical components 44 of the male pelvic anatomy 42. Based upon the geometric model 40, mathematical equations can be obtained that relate to the shape of the torso 42 and the anatomical components 44 within the torso 42. To accurately model the anatomy of the pelvis, changes (e.g., in density) along all three dimensions of the pelvis are accounted for in equations. Therefore, while FIG. 5 illustrates a two-dimensional representation of the geometry of the torso 42, VPs, like those shown in FIGS. 2-4, are three-dimensional and the mathematical equations that describe the phantoms are also three-dimensional. Such three-dimensional modeling accounts for variances in the shape and/or positioning of the torso 42 and the anatomic components 44 along the cranial-caudal axis (here, along the z-axis).

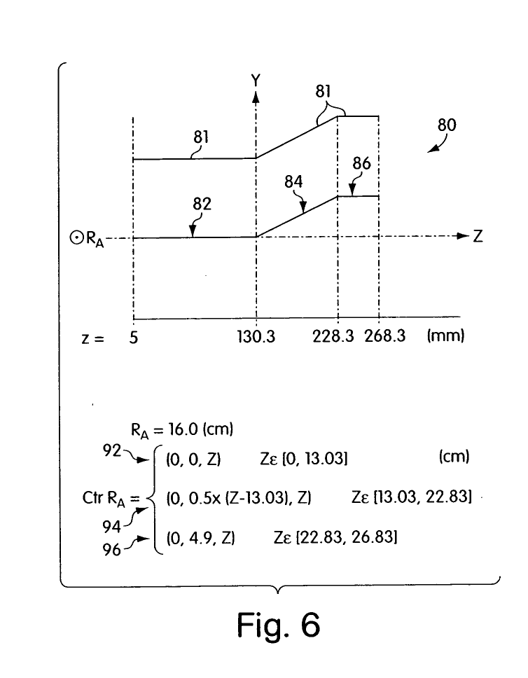

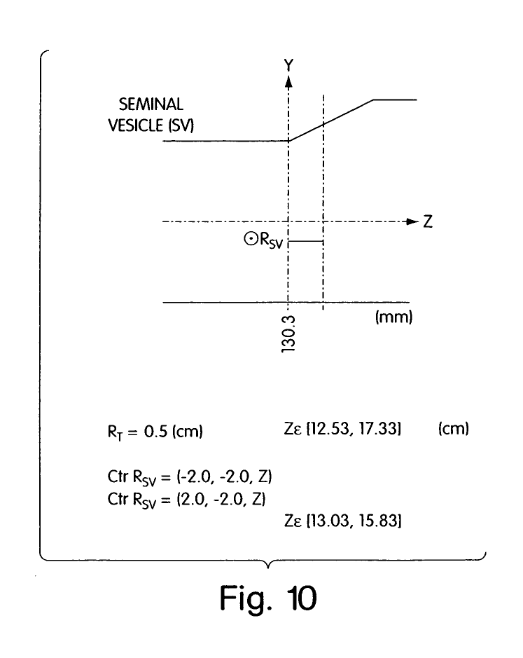

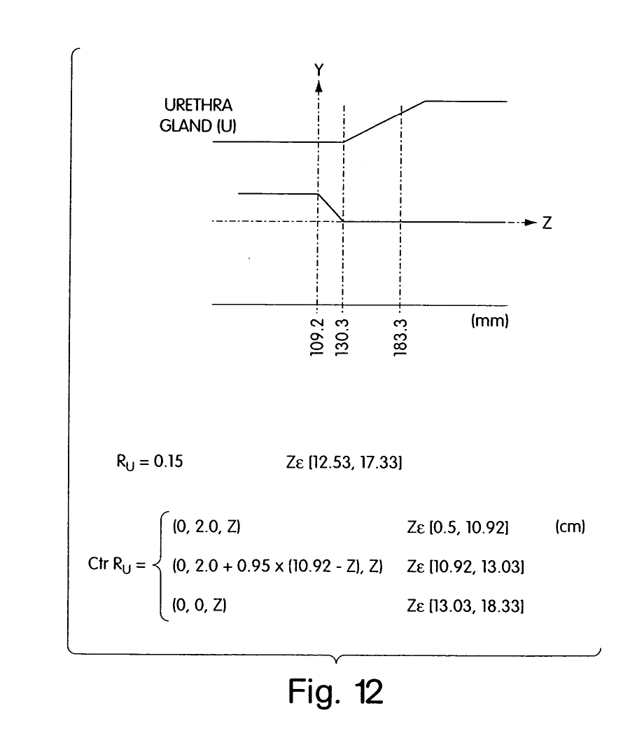

FIGS. 6-12 illustrate geometric variance of the torso 42 shown in FIG. 5 and the anatomical components 44 in the torso 42 along the along the cranial-caudal axis. FIGS. 6-12 also show the geometric modeling and associated equations relating to the torso 42 and the anatomic components 44.

Referring to FIG. 6, a graph 80 indicates the deviation of the center 68 of the torso 42 along the cranial-caudal axis, here the z-axis, and the corresponding change in height of the torso 42. A trace 81 indicates the height of the torso 42 as a function of z. This trace follows the pattern of the change in position of the center 68 of the circle 48 as the radius of the circle 48 does not change as a function of z. A portion 82 indicates that along the z-axis from the origin up to a distance of 130.3 mm away from the origin, the center 68 of the torso 42 does not deviate along the y-axis. This lack of deviation is reflected in a mathematical equation 92 that reflects the geometric positioning of the center 68 of the torso 20 with respect to the origin. A portion 84 of the graph 80 illustrates that along the z-axis between a distance of 130.3 mm away from the origin and a distance of 228.3 mm away from the origin, the center point 68 of the torso 42 moves upwardly in the y direction (i.e., the center point 68 of the torso 42 is elevated with respect to the origin). A mathematical equation 94 reflects this geometric deviation of the center 68 of the torso 42 with respect to the origin. A portion 86 of the graph 80 illustrates that along the z-axis between a distance of 228.3 mm away from the origin and a distance of 268.3 mm away from the origin, the center point 68 does not deviate in the y direction, but remains in an elevated position. A corresponding mathematical equation 96 reflects this geometric positioning of the center 68 of the torso 42 with respect to the origin. Throughout the z-axis distance shown, the radius of the circle 48 is a constant 16.0 cm.

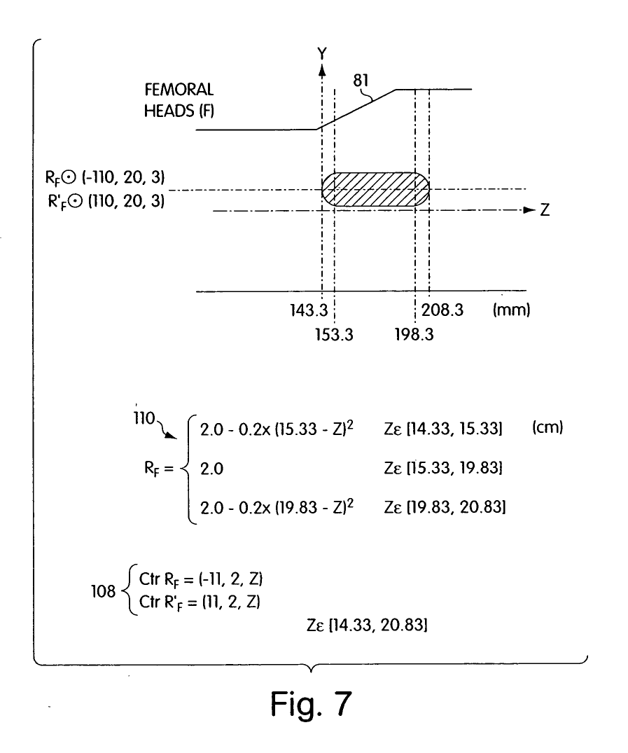

FIGS. 7-12 illustrate geometric modeling and associated equations relating to other of the anatomic components 44. FIGS. 7-12 show the y-dimension span of the various components 44 over distances in the z-direction. These figures show changes in the radii and/or centers of the circles of the components 44 in the model 40 as a function of the z-axis. The figures also provide the corresponding equations for the center and radius magnitude for the various components 44. The particular forms and equations of the components are exemplary only and not limiting.

FIG. 7 shows the changes in the radii of the modeled femoral heads 54, 56 along the z-axis. As shown, the locations of the femoral heads 54, 56 relative to the z-axis do not change as a function of distance in z. The heads 54, 56 are centered at +20 mm in the y-direction, and at −110 and +110 mm in x, respectively, as indicated by equations 108. The radii of the heads 54, 56 vary as functions of z as indicated by equations 110. The heads 54, 56 are simulated as cylinders, bounded on either end by hemispheres. The trace 81 indicates the top of the torso 42 as a function of z.

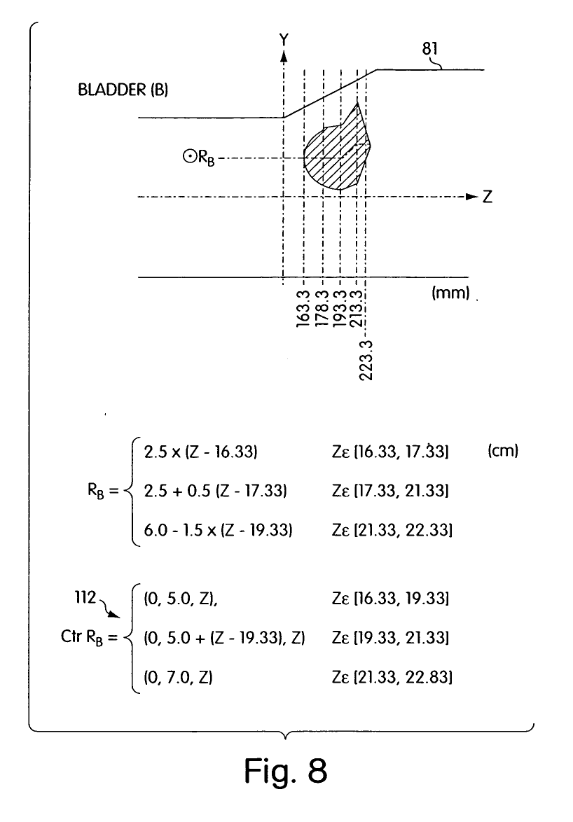

FIG. 8 shows the changes in the radii of the modeled bladder 58 along the z-axis. As shown, an equation set 112 mathematically models changes in the location of a center point 114 of the bladder 58 relative to the y-axis as a function of position along the z-axis. FIG. 8 does not reflect the deformation of the bladder 58 due to interference with the prostate 60. In practice, the height in the y-direction of the bladder 58 would be made to track the y-displacement of the prostate 60.

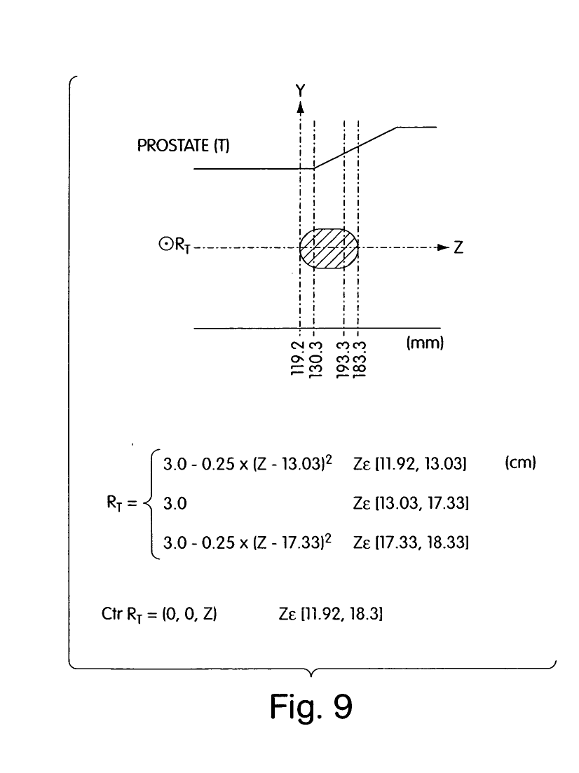

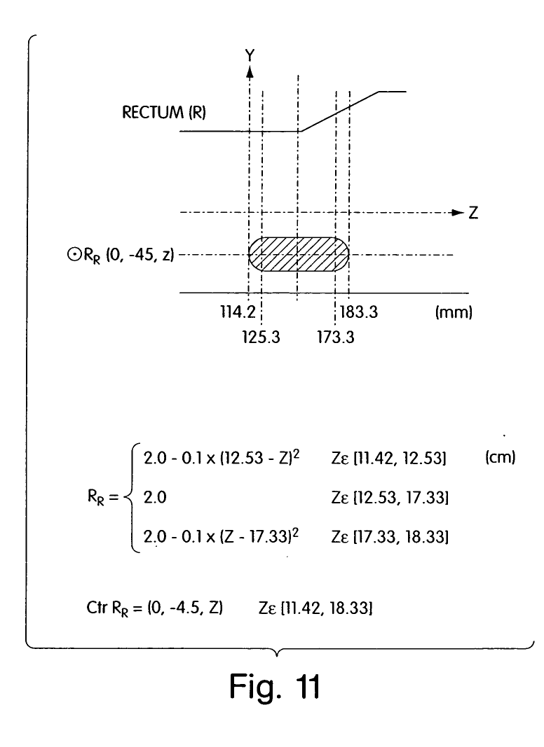

FIGS. 9-12 show similar changes in corresponding radii and centers for other of the components 44. FIGS. 9-12 show these changes for the prostate 60, the seminal vesicles 64, 66, the rectum 62, and the urethra 72. FIG. 11 does not reflect the deformation of the rectum 62 due to interference with the prostate 60. In practice, the height in the y-direction of the rectum 62 would be made to track the y-displacement of the prostate 60.

The model 40 is exemplary and not limiting. Other shapes, sizes of shapes, or spatial relationships of the shapes may be used. The dimensions, positions, and functions of position shown and described above are examples only, with other values and functions being acceptable. Further, various densities can be used for the components. Additionally, models are preferably made for other parts of the human body, e.g., the head. The components 44 can vary in other directions (e.g., in x or y) and appropriate equations can be provided for such variations.

Referring to FIG. 13, a real anthropomorphic phantom 210 can be produced in accordance with the dimensions of the VPs. The VP is used as the guide for producing the RP. The data (e.g., equations, density specifications, effective atomic number specifications, etc.) can be provided, e.g., electronically, to a phantom manufacturer, e.g., The Phantom Laboratory, Inc. of Salem, N.Y., for production of the RP 210 according to the geometrically modeled anatomy of the VP. The manufacturer can produce an RP using the geometric equations provided and using materials to match or approximate the desired properties, e.g., densities and effective atomic numbers. The VP parameters may be supplied with the RP 210, e.g., on a computer-readable medium such as a CD-ROM. Preferably, the VP is produced with densities and effective atomic numbers that the manufacturer can produce in mind. The manufacturer can use a variety of materials for the RP 210. For example, the manufacturer can use an isocyanate rubber or synthetic muscle equivalent such as A-150 plastic. Preferably, the RP 210 does not use actual bone material or other human tissue. The materials used in the phantom 210 are preferably of lower cost than using actual human components such as bone, and provide for a more consistent density over time than human materials. The materials of the phantom 210 are preferably resistant to dessication and decay, or at least more so than human materials such as bone.

The RP 210 approximates the VP in a radiation-relevant manner, i.e., such that the RP 210 and the corresponding VP 40 will affect applied radiation similarly. Thus, soft-tissue organs with similar properties may be formed as a common material and not individually formed, but represented as a block of material with individual organs being corresponding volumes within (portions of) the block.

The RP 210 is configured to accommodate one or more radiation detectors. The RP 210 includes several holes 212 (including non-circular shapes such as slots) that are sized and shaped to receive radiation detectors such as ionization chambers. Preferably, the detectors are configured to rotate and the holes 212 are configured to permit rotation of the detectors within the holes 212 such that the detectors can be moved to different angles relative to the incident beam. Thus, beam-geometry-dependent measurements can be taken to remove/reduce measurement artifacts and improve estimates of measured radiation dose.

Different inserts can be designed to accommodate different types of radiation detectors. For example, inserts can be designed for ion chambers, gel detectors, diodes, films, etc., so that these detectors can be inserted into the phantom 210. Inserts can accommodate detectors, e.g., film, gel, thermoluminescent detectors, that need not have a connection to an outside device. Inserts may also holes leading to the outside of the RP 210 to accommodate detectors that are connected to external devices. For example, a disc-shaped insert 220 shown in FIG. 13 is configured to the shape and size of the detector and can be rotated to provide different orientations for the detector. The access holes 212 for the insert 220 are preferably coplanar and dowels 222 are provided to fill the unused access holes 212. Inserts may also be configured to accommodate multiple detectors, with spacers to separate the detectors, e.g., arranged in an array.

The RP 210 includes the modeled organs as well as substances in the organs, such as gases or fluids. Passages 214 are provided for receiving tubes 216 that can deliver gases and/or fluids to desired regions in the phantom 210 that are accessible via the passages 214. The gas is provided in the desired organ/region at a desired pressure, e.g., to simulate a condition of a patient. Fluid may be provided with characteristics and in an amount reflecting the condition of the patient, e.g., fluid in the lungs reflecting pneumonia. One or more fluids and/or one or more gases can be provided to the same cavity in the phantom 210. Different compositions of substances can be put into the phantom to achieve a desired density and/or atomic number in the cavity receiving the substance(s).

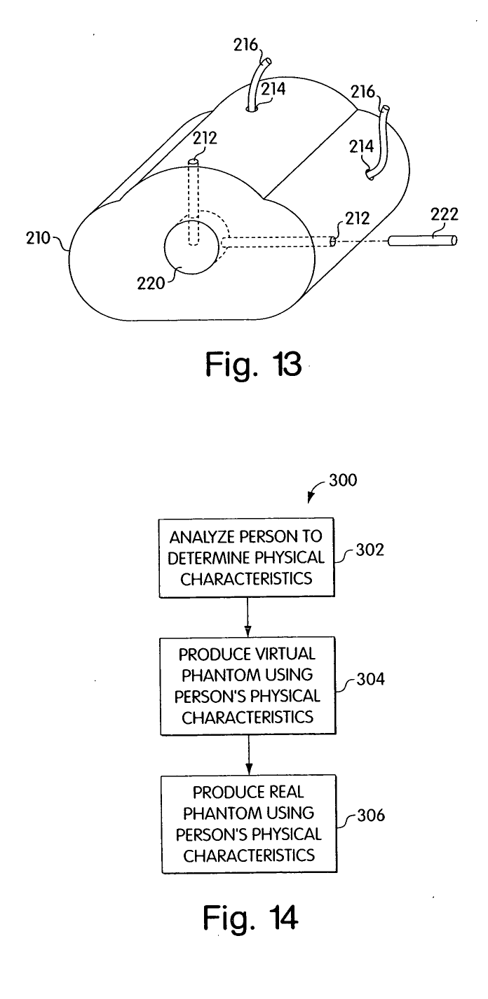

In operation, referring to FIG. 14, with further reference to FIGS. 1-13, a process 300 for producing the VP 40 and the corresponding RP 210 includes the stages shown. The process 300, however, is exemplary only and not limiting. The process 300 may be altered, e.g., by having stages added, removed, or rearranged.

At stage 302, a person is studied to determine it's the person's physical characteristics. A human being or a portion thereof is studied to determine sizes, shapes, and densities of the human's anatomy as well as the spatial relationships between the components of the anatomy. Here, at least the abdomen of a human is studied to determine the corresponding anatomy.

At stage 304, the VP 40 is produced based on the analysis from stage 302. The organs and other components of the person determined in stage 302 are simplified to well-known geometric shapes. Equations are produced to represent the sizes, shapes, and locations of the components of the anatomy. Densities are assigned to the components. Physical attributes of the components (e.g., size, shape, density, etc.) may vary as a function of location of the components. The densities assigned may depend upon the densities that a manufacturer of the RP 210 can produce.

At stage 306, the RP 210 is produced by a phantom manufacturer. The characteristics of the VP 40 are provided, e.g., sent electronically or at least in electronic form, to a phantom manufacturer. The manufacturer selects materials for each of the components depending upon the desired densities. The various components are produced (e.g., molded) in the desired shapes and assembled in the indicated relationships. Multiple RPs 210 may be manufactured according to the same specifications and provided to multiple radiation facilities.

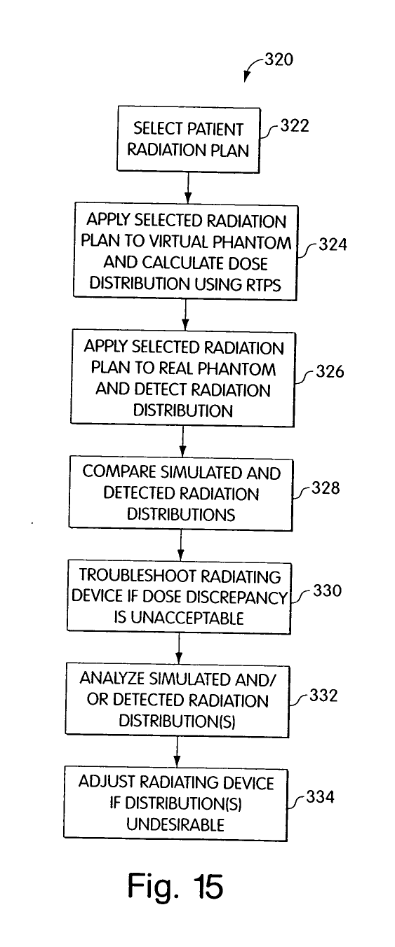

In operation, referring to FIG. 15, with further reference to FIGS. 1-13, a process 320 for accepting and commissioning the radiating device 12 using the system 10 includes the stages shown. The process 320, however, is exemplary only and not limiting. The process 320 may be altered, e.g., by having stages added, removed, or rearranged.

At stage 322, a patient radiation plan is selected with corresponding radiation parameters for the radiating device 12. The plan may be a test plan, e.g., that is preprogrammed into the treatment planning system 18 or the radiating device, or may be a custom plan, e.g., selected by a qualified radiation physicist.

At stage 324, the selected radiation plan is “applied” to the VP 40 (or other VP as desired). The treatment planning system simulates the application of the selected radiation plan to calculate a dose distribution in the VP 40 using the RTPS 18. Indicia of the induced, simulated radiation distribution can be provided in various forms, e.g., graphs, data sets, tables, dose volume histograms (graphs of volume as a function of dose), etc. For example, the indications can be three-dimensional transparent surface renderings of PTV, OAR, and/or isodose surfaces. The indications may also be in the form of multiple two-dimensional plots of isodose distributions.

At stage 326, the selected radiation plan is applied to the RP 210. The RP 210 is placed in the system 10 and the radiating device 12 radiates the phantom 210 according to the selected radiation plan. One or more radiation detectors are placed in the phantom 210 to detect radiation. The detector(s) may be moved to reduce artifacts and improve detection. The detector(s) may also be moved to determine radiation in two or three dimensions versus at single points, with the phantom 210 being configured to permit movement in two or three dimensions. Preferably, radiation is detected near a region intended to receive the most radiation, e.g., in a region of high dose gradient. The detected radiation intensity(ies) is(are) stored for analysis and/or comparison.

At stage 328, the simulated and detected radiation distributions are compared. Data regarding the simulated and actual radiations distributions from similar locations in the virtual and real phantoms 40, 210 are compared to each other to determine the amount(s) of disparity between the simulated and detected radiation distributions. In particular, the comparison looks for overdosing healthy tissue and underdosing targeted tissue.

At stage 330, troubleshooting is performed on the radiating device 12 and/or the treatment planning system 18 if the calculated and actual dose distribution discrepancies (of the compared portion(s) of the dose distributions) differ undesirably. For example, disparities within threshold amounts may be accepted without troubleshooting while disparities greater than a threshold/tolerance require troubleshooting before the radiating device 12 will be accepted/commissioned. Various types of disparities may be analyzed, e.g., average intensity difference, peak intensity difference, intensity difference in one or more regions such as regions of high dose gradient, etc. Further, comparisons can be made of dose volume histograms, e.g., to reveal dose discrepancies that may be described in volumes of regret. Disparities may be caused by and/or indicative of various issues with the radiating device 12 such as gantry sag, table wobble, errors with collimators, etc. The troubleshooting is performed to attempt to correct errors with the radiating device 12. For example, integrities of the device 12 may be investigated, including collimator motion, gantry sag, table axis wobble, beam quality and stability, etc. Also, integrities of the treatment planning system 18 may be investigated including beam modeling, dose calculation accuracy, etc. If troubleshooting is unable to find and/or correct errors inducing the disparity between simulated and actual radiation distribution, then the device 12 is rejected, e.g., not accepted or commissioned.

At stage 332, the simulated and/or detected radiation distributions is/are analyzed to determine whether it/they provide a desired distribution and/or distributions. Either radiation distribution is analyzed to determine whether the distribution meets acceptable criteria such as whether a targeted region in the phantom receives sufficient radiation for treatment and whether surrounding regions do not receive undesirably high amounts of radiation.

At stage 334, the radiating device 12 is adjusted based upon the analysis of the radiation distribution(s) in either or both of the virtual and real phantoms 40, 210. For example, radiation parameters may be adjusted to change the focus of the radiation, the contours of isodose surfaces, etc. If adjustments can be made to provide desirable radiation distributions, then the device 12 may be accepted/commissioned, and if not then the device 12 is not accepted. Further, analysis may be performed to determine if the radiation distribution for the device under test meets standard criteria for the radiation plan and phantom used. This can be done to compare the particular radiating device 12 to other radiating devices, e.g., at other facilities. This helps to provide standardizing of radiating devices across multiple facilities to help ensure that different devices provide similar distributions for similar radiation plans. Thus, results of treatments at different facilities can be compared and used as part of large studies such as clinical trials.

As mentioned above, the process 320 can be modified in various ways. For example, the troubleshooting stage 330 can be performed after the analysis stage 332. Further, the analysis and adjusting stages 332, 334 may be eliminated if the radiating device 12 is rejected at stage 330. Similarly, the comparison stage 328 and the troubleshooting stage 330 may be eliminated if the analysis and adjusting stages 332, 334 are performed first and the device 12 is rejected.

Further, the process 320 can be modified for patient-specific quality assurance (QA). For example, for patient-specific QA, the radiation plan selected at stage 322 may be a treatment plan developed for a particular patient and that patient's particular needs. Here, a radiation oncologist may specify an amount of radiation to be directed toward a target or clinical tumor volume (CTV) and radiation exposure limits for healthy surrounding tissue. The treatment planning system 18 can use this information to develop a radiation plan including radiation parameters for the radiating device to best implement the desired radiation distribution. Also, the analysis and adjusting stages 332, 334 may be eliminated. Such QA may be performed before each patient.

Further still, the process 320 may be modified to compare the simulated dose distribution with a dose distribution calculated using Monte Carlo analysis. Exemplary Monte Carlo models include EGS4 (electron gamma shower) and MCNP (Monte Carlo Neutron Proton). In the case of Monte Carlo analysis, a dose distribution calculated using Monte Carlo radiation transport analysis is used in place of the distribution in the RP 210. Thus, at stage 328 the dose distribution calculated by the RTPS 18 is compared against a dose distribution calculated using Monte Carlo radiation transport analysis. At stage 330, troubleshooting is performed if the discrepancy between the RTPS-calculated distribution and the Monte Carlo-calculated distribution differ unacceptably. Further, at stage 332, the distributions calculated by the RTPS 18 and the Monte Carlo analysis are analyzed, and at stage 334 adjustments may be made if either of the calculated distributions is undesirable.

Other embodiments are within the scope and spirit of the appended claims. For example, due to the nature of software, functions described above can be implemented using software, hardware, firmware, hardwiring, or combinations of any of these. Features implementing functions may also be physically located at various positions, including being distributed such that portions of functions are implemented at different physical locations. Further, while the above description specifically discussed use of phantoms to represent humans, the phantoms could model and be used for analysis regarding other objects including other animals, inanimate objects, etc.

1. A real, physical radiation phantom for simulating a portion of a human being, the phantom comprising:
a body portion providing an analytic outer shape of the phantom, the outer shape being similar to a shape of at least a portion of the human being, the body portion having a first physical characteristic of a first value similar to a second value of the first physical characteristic corresponding to human soft tissue; and
at least one internal component disposed in the body, the internal component having an analytic shape approximating an internal portion of human anatomy and having a third value of the first physical characteristic different from the first value.

2. The phantom of claim 1 wherein the first physical characteristic is one of density and effective atomic number.

3. The phantom of claim 2 wherein the at least one internal component is configured to approximate a shape of a human bone and the third value is one of an average density of the human bone and an effective atomic number of the human bone.

4. The phantom of claim 1 wherein the at least one internal component is configured to approximate a shape of a human bone, at least a part of the at least one internal component including multiple portions configured to simulate different layers of bone, the multiple layers including a first portion having a first density and a first atomic number similar to a density and atomic number of an outer, relatively harder layer of human bone and a second portion inside the first portion and having a second density and a second atomic number similar to a density and atomic number of an inner, relatively softer layer of human bone.

5. The phantom of claim 1 wherein the at least one internal component comprises multiple internal components of shapes approximating internal components of the human being and having corresponding densities and atomic numbers similar to the corresponding internal components of the human being.

6. The phantom of claim 5 wherein the multiple internal components have densities and atomic numbers similar to at least one of bone, soft tissue, lung, and fat.

7. The phantom of claim 1 wherein the phantom provides at least one hole configured to receive a radiation detector and sized to permit rotation of the radiation detector inside the phantom.

8. The phantom of claim 1 wherein the phantom provides at least one passage extending from an outer surface of the body to a cavity defined inside the phantom, the passage being configured to convey at least one of gas and liquid to the cavity.

9. A real-virtual phantom system comprising:
an anthropomorphic virtual phantom that includes analytic shapes representing human anatomical parts; and
an anthropomorphic real, physical phantom that approximates the virtual phantom in a radiation-relevant manner with a first material that simulates human soft tissue and at least one second material that simulates other tissue that affects radiation differently than soft tissue, the at least one second material having an analytic shape that approximates a corresponding portion of human anatomy.

10. The system of claim 9 wherein corresponding portions of the virtual and real phantoms have similar densities and atomic numbers.

11. The system of claim 10 wherein the densities and atomic numbers correspond to at least one of bone, soft tissue, lung, and fat.

12. The system of claim 9 wherein the real phantom provides at least one hole configured to receive a radiation detector and sized to permit rotation of the radiation detector inside the phantom.

13. The system of claim 9 wherein the real phantom provides at least one passage extending from an outer surface of the real phantom to a cavity defined inside the real phantom, the passage being configured to convey at least one of gas and liquid to the cavity.

14. The system of claim 9 wherein the anthropomorphic virtual phantom comprises numerical expressions disposed on a computer-readable medium.

15. The system of claim 9 wherein the analytic shapes of human anatomical parts of the anthropomorphic virtual phantom represent human anatomical parts that are high-probability targets for radiation therapy.

16. A method of using a first virtual radiation phantom, the method comprising:
calculating a first radiation distribution from a first radiating device in the first virtual radiation phantom, the first virtual radiation phantom modeling human anatomical components as analytic shapes; and
comparing indicia of the first radiation distribution with information from a second radiation distribution.

17. The method of claim 16 wherein the information from the second radiation distribution is information of radiation detected in a first physical phantom configured to approximate physical characteristics modeled by the first virtual radiation phantom in a radiation-relevant way.

18. The method of claim 17 wherein the first virtual radiation phantom and the physical phantom are substantially similar to a second virtual radiation phantom and a second physical phantom used with a second radiating device as part of a clinical test.

19. The method of claim 17 further comprising troubleshooting the first radiating device if appropriate as determined from the comparing.

20. The method of claim 19 further comprising radiating a human patient and providing information associated with radiating the human patient to a repository of information for a clinical test.

21. The method of claim 20 wherein the radiating comprises radiating the patient with an IMRT device.

22. The method of claim 17 further comprising performing an analysis on at least one of the indicia of the first radiation distribution and the information from the second radiation distribution and adjusting radiation parameters of the first radiating device, if appropriate, based upon the analysis.

23. The method of claim 16 wherein the information from the second radiation distribution is information calculated using a Monte Carlo radiation transport analysis.
